(19) United States
(12) Patent Application Publication
(10) Pub. No.: US 2020/0038644 A1
(43) Pub. Date: Feb. 6, 2020

(54) Venous Access Port Assembly With Radiopaque Indicia

(71) Applicant: Medical Components, Inc., Harleysville, PA (US)

(72) Inventors: Kenneth M. Zinn, Westport, CT (US); Raymond Bizup, Feasterville, PA (US); Kevin Sanford, Chalfont, PA (US); Timothy M. Schweikert, West Chester, PA (US)

(21) Appl. No.: 16/600,646

(22) Filed: Oct. 14, 2019

Related U.S. Application Data
(63) Continuation of application No. 11/725,287, filed on Mar. 19, 2007.
(60) Provisional application No. 60/852,591, filed on Oct. 18, 2006.

(51) Int. Cl. A61M 39/02

(52) U.S. Cl. CPC A61M 39/0208; A61M 2039/0238; A61M 2039/0072; A61M 2039/0223; A61M 2039/0211

(57) ABSTRACT

A venous access port assembly (10) having a housing base (28) with a discharge port (16), a septum (14) and a cap (48). An interior reservoir (22) is defined by a well (30) in the housing base and a bottom (62) of the septum, and a passageway (20) extends from the reservoir through the discharge port (16). The housing base (28) is provided with radiopaque markings (60) on the bottom wall (44) thereof, including radiopaque indicia (70) that identify an attribute of the assembly (10) after its implantation and clearly appear on an X-ray of the patient in a manner informing the radiologist or technologist and the medical practitioner of that particular attribute.

### CROSS REFERENCE TO RELATED APPLICATIONS

This application claims priority from Provisional U.S. Patent Application Ser. No. 60/852,591 filed Oct. 18, 2006.

### FIELD OF THE INVENTION

This relates to the field of medical devices and more particularly to venous access ports for the infusion of fluids into the patient and/or withdrawal of fluids from the patient.

### BACKGROUND OF THE INVENTION

Venous access ports for the infusion and/or withdrawal of fluids from a patient are well-known, secured to the proximal end of an implanted catheter. These ports are typically used for drug infusion or for withdrawal of small amounts of blood, where large flows of fluid are not required. The ports are assemblies of a needle-impenetrable housing with a discharge port in fluid communication with the catheter and the reservoir within the port housing, and provide a subcutaneous self-sealing septum that defines an access site for multiple needle sticks through the covering skin tissue of the patient, through the septum and into the reservoir, without the need to continuously search for new access sites. Examples of such ports are disclosed, for example, in U.S. Pat. Nos. 4,704,103; 4,762,517; 4,778,452; 5,185,003; 5,213,574 and 5,637,102.

It is desired to provide a venous access port assembly that provides for a radiologist, radiology technologist, nurse and ultimately a medical practitioner to be able to discern an important property of the port assembly after the port assembly has been implanted into a patient.

### BRIEF SUMMARY OF THE INVENTION

The present invention is related to a venous access port having a housing and a septum, providing an interior reservoir and a passageway extending from the reservoir through a stem of a discharge port to establish fluid communication with a proximal end of a catheter lumen to which the port assembly is secured prior to placement of the assembly into a patient. The port may optionally have more than one reservoir and associated septum. The invention is the application of radiopaque indicia onto a venous access port that is discernible under X-ray examination to provide information concerning the nature or key attribute of the venous access port, so that the practitioner, subsequent to the date of implantation thereof, can determine that nature or key attribute under X-ray examination. One such key attribute in particular would be for example that the venous access port is rated to be used for power injection such as of contrast fluid, wherein for example the letters “CT” (for “computed tomography”, or “contrast enhanced computed tomography”) would be provided that are of radiopaque material, optionally positioned within radiopaque circles. The attribute in this example is the property of the port's being adapted to withstand high pressures that are used for injection of contrast fluid into a patient, and the letters “CT” would be understood in medical practice to indicate that the port is suitable for the high pressure injection of contrast fluid. The radiopaque indicia could for example be applied in a mirror-image orientation on the bottom housing surface, and would appear on the X-ray as right-side up and easily readable by the radiologist, technologist or practitioner.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is an isometric view of the venous access port of the present invention;

FIG. 2 is a plan view of the port of FIG. 1;

FIGS. 3 and 4 are cross-section views of the port of FIGS. 1 and 2 taken along lines 3-3 and lines 4-4 of FIG. 1, respectively;

FIG. 5 is an isometric view of the needle-impenetrable housing base of the venous access port of FIG. 1;

FIGS. 6 and 7 are transverse cross-sectional and longitudinal cross-sectional views of the housing base of FIG. 5;

FIG. 8 is an isometric view from below of the housing base of FIGS. 6 and 7, showing the radiopaque indicia applied on the housing base bottom surface; and

FIGS. 9 and 10 are bottom and top views of the housing base of FIG. 8 having radiopaque indicia thereon, with the top view being analogous to the X-ray view of the venous access port by the radiologist, and the indicia being shown in dashed lines in FIG. 10

### DETAILED DESCRIPTION OF THE INVENTION

Certain terminology is used herein for convenience only and is not to be taken as a limitation on the present invention. The terms “distal” and “proximal” refer, respectively, to directions closer to and away from the insertion tip of a catheter in an implantable catheter assembly. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

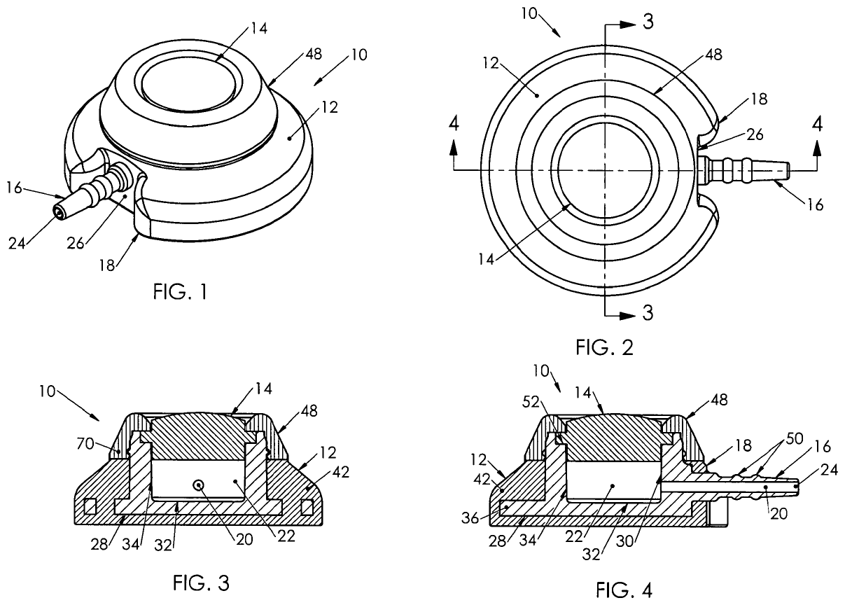

Venous access port assembly 10 of FIGS. 1 to 4 includes a housing 12 and a septum 14, with a discharge port 16 extending from a distal end 18 of the port assembly 10 to be attached securely and sealingly to the proximal end of a catheter (not shown). One such venous access port assembly is disclosed in U.S. patent application Ser. No. ______ filed ______ (corresponding to U.S. Provisional Patent Application Ser. No. 60/801,523 filed May 18, 2006). A passageway 20 extends from the interior reservoir 22 to the distal tip opening 24 of discharge port 16. A recess 26 is seen to be provided along both sides of discharge port 16, facilitating insertion of the discharge port 16 into the catheter lumen and providing a clearance for a locking sleeve or clamp (not shown) utilized to compress the catheter lumen wall against the exterior surface of the discharge port 16 for assured sealed connection of the catheter with the port assembly 10.

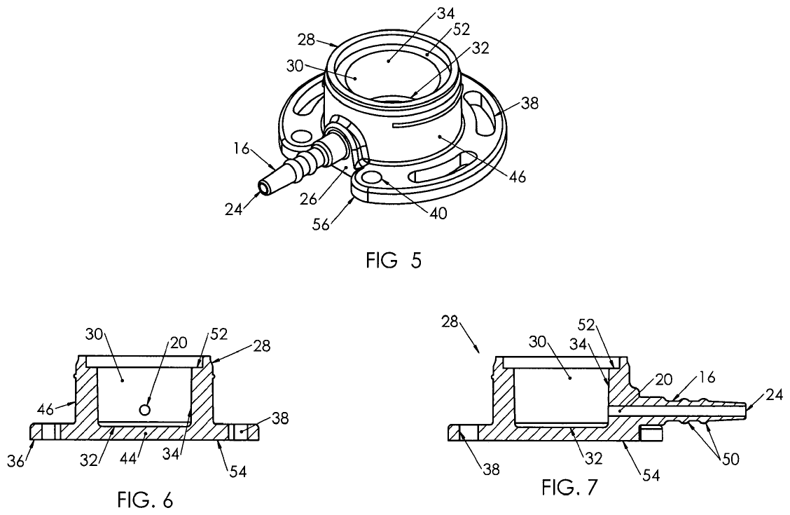

With reference now to FIGS. 3 to 7, the interior of the port assembly 10 is shown to provide an interior reservoir 22. Housing 12 is shown to include a housing base 28 of needle-impenetrable material that includes a well 30 having a bottom floor 32 and side walls 34 that define the interior reservoir 22 beneath septum 14. Bottom floor 32 may be convex or elevated (not shown) toward the center of the reservoir, if desired. Housing base 28 includes a base flange 36 extending radially outwardly from the bottom of well 30, and base flange 36 includes openings 38,40 that se o enable suturing to the patient upon placement of the venous access port and the attached catheter into the patient.

As shown in FIGS. 3 and 4, a skirt 42 is overmolded about housing base 28 and may be of silicone elastomer. It is seen that skirt 42 encapsulates the outer surfaces of the bottom wall 44 and the bottom portion of the side walls 46 of housing base 28, and is shown to fill in the suture holes 38,40; but since the material is silicone elastomer, suturing is possible since the suturing needle can easily be inserted through the material of skirt 42 and through the suture holes, and thereafter the filled openings provide minimal opportunity for ingrowth of patient tissue into the openings.

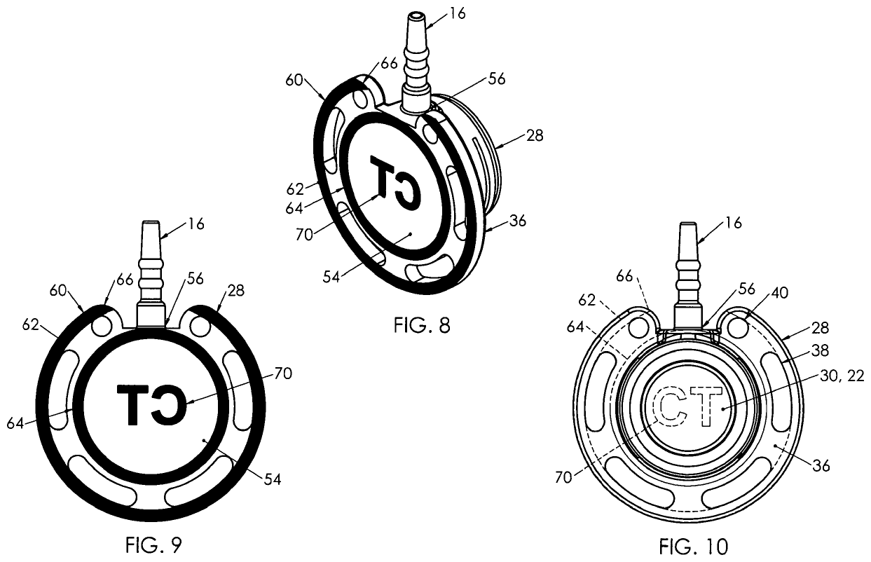

Also seen in FIGS. 1 to 4 is cap 48, which secures to housing base 28 to in turn secure septum 14 in position in the port assembly 10. Preferably, skirt 42 is insert molded onto base flange 36 of housing base 28 after cap 48 is secured to the upper portion of housing base 28 to secure the septum in position. It is seen in FIGS. 4 and 7 that discharge port 16 is integral with housing base 28 as is preferable. Discharge port 16 is shown to have a pair of annular ridges 50 that facilitate with the mechanical connection of the catheter proximal end with the port assembly 10. Housing base 28 includes a septum seat 52 extending into the top of well 30, into which a flange of the septum will be seated, preferably under radially inward compression. Housing base 28 has a bottom outer surface 54.

Radiopaque markings 60 of the present invention are shown in FIGS. 8 to 10. A larger outer circle 62 is seen provided on the outermost periphery of bottom base surface 54, and a smaller inner circle 64 is seen provided within the area circumscribed by the suture openings 38 and holes 40 through base flange 36. Adjacent to discharge port 16, a recess 56 is provided in the skirt of the housing base to provide a clearance for use of a connection sleeve that will be used to secure the catheter (not shown), and outer circle 62 is shown to have a gap 66 at the recess. Outer and inner circles or rings 62,64 circumscribe radiopaque indicia 70.

Radiopaque indicia 70 are provided on bottom outer surface 54 within the region directly beneath the reservoir and septum. In the example shown, indicia 70 comprise the letters “CT” (FIG. 10) representing the term “computed tomography.” The meaning of this particular example of indicia is that the venous access port assembly 10 is rated for high pressure injection such as is necessary for infusion into the patient of contrast medium that is used in computed tomography. Other indicia may of course be used that indicate some other attribute or characteristic of the venous access port assembly. The radiopaque markings and indicia would appear on an X-ray of the patient, and the indicia are provided in a mirror-image orientation on the bottom outer surface of the housing base (FIGS. 7 and 8) so that the indicia would appear as “CT” when the X-ray is viewed (FIG. 9), easily discerned by the radiologist or technologist. Centering of the indicia within the region (identified as “30,22” in FIG. 10) directly beneath the reservoir and septum minimizes any obscuring by the structure of the venous access port assembly, and the indicia may also be easily discernible should the port assembly be at an angle from the horizontal plane of the X-ray; the outer and inner circles 62,64 would appear oval or elliptical should the port assembly be at such an angle. Gap 66 in outer circle 62 would also appear and would indicate the location of the discharge port stem 16.

The radiopaque markings may constitute marking fluid that is embossed or imprinted or otherwise applied onto the surface of the housing base 28, such as black radiopaque ink Part No. C11002 Rev A formulated by Creative Imprinting of Erie, Pa., from Marabu Tampapur TPU 910 clear with tungsten added, available from Marabuwerke GmbH & Co. KG of Stuttgart, Germany, and may be applied on plasma-treated surfaces. At least the housing base 28, the septum 14 and the skirt 42 are of radiotransparent or radiolucent material as is well known in implanted medical devices, and the housing base may be molded of polysulfone resin.

The radiopaque markings may alternatively applied to the inwardly facing surface of the bottom wall of the housing base, or may constitute foil or film (such as a decal) of radiopaque material embedded within the housing base, these alternatives not being shown in the drawings.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1-11. (canceled)

12. A venous access port assembly, comprising:
a housing comprising a housing base defining at least one reservoir with a passageway extending from the at least one reservoir through a stem of a discharge port, wherein at least a portion of the housing comprises material that is at least one of radiotransparent and radiotranslucent; and,
at least one radiopaque marking that is at least one of provided in a portion of the housing and provided on the portion of the housing.

13. The assembly recited in claim 12, wherein the at least one radiopaque marking further includes radiopaque indicia that is at least one of provided in the portion of the housing and provided on the portion of the housing base.

14. The assembly recited in claim 12, wherein the at least one radiopaque marking comprises marking fluid that is applied onto a surface of the housing base.

15. The assembly recited in claim 14, wherein the marking fluid is embossed or imprinted onto the surface of the housing base.

16. The assembly recited in claim 12, wherein the housing base has a bottom outer surface and the at least one radiopaque marking is at least one of provided in the bottom outer surface and provided on the bottom outer surface.

17. The assembly recited in claim 12, wherein the at least one radiopaque marking comprises foil.

18. The assembly recited in claim 12, wherein the at least one radiopaque marking comprises film.

19. A venous access port assembly, comprising:
a housing comprising a well having a bottom floor and side walls that define an internal reservoir, the housing further comprising a housing base made of needle-impenetrable material and a discharge port leading to a passageway that extends from the internal reservoir; and,
radiopaque material that is at least one of provided in a portion of the housing and provided on the portion of the housing.

20. The assembly recited in claim 19, wherein the bottom floor is convex.

21. The assembly recited in claim 19, wherein the bottom floor is elevated toward a center of the internal reservoir.

22. The assembly recited in claim 19, wherein the radiopaque material is at least provided on an inwardly facing surface of the side walls.

23. The assembly recited in claim 19, wherein at least a portion of the housing is at least one of radiotransparent and radiotranslucent.

24. The assembly recited in claim 19, further comprising at least one of a radiotransparent material molded around the housing base and a radiotranslucent material molded around the housing base.

25. A venous access port assembly, comprising:
a housing comprising:
a housing base defining at least one reservoir;
a discharge port extending from a portion of the housing; and,
a passageway extending from the at least one reservoir to the discharge port; and,
at least one marking that is at least one of provided in a portion of the housing and provided on the portion of the housing, the at least one marking being discernible under x-ray examination and providing information regarding at least one of a location of the discharge port and that the access port assembly is rated for power injection.

26. The assembly recited in claim 25, wherein the at least one marking comprises radiopaque material.

27. The assembly recited in claim 25, wherein at least a portion of the housing comprises a material that is at least one of radiotransparent and radiotranslucent.

28. The assembly recited in claim 25, wherein the housing base comprises a septum seat.

29. The assembly recited in claim 28, further comprising a needle-penetrable septum placed within the reservoir.

30. The assembly recited in claim 29, further comprising skirt overmolded about the housing base.

31. The assembly recited in claim 30, wherein at least one of the housing base, the septum, and the skirt is at least one of radiotransparent and radiotranslucent.
